SEQUENCE LISTING (22 sequences)

SEQ ID NO: 1 - DNA, 49 bp, Artificial sequence
  synthetic
gcgagcacagaattaatacgactcactataggtttttttttttttttvn

SEQ ID NO: 2 - DNA, 22 bp, Artificial sequence
  synthetic
gcgagcacagaattaatacgac

SEQ ID NO: 3 - DNA, 22 bp, Artificial sequence
  synthetic
tgaggtagtaggttgtatagtt

SEQ ID NO: 4 - DNA, 22 bp, Artificial sequence
  synthetic
tgaggtagtaggttgtgtggtt

SEQ ID NO: 5 - DNA, 23 bp, Artificial sequence
  synthetic
caaagtgcttacagtgcaggtag

SEQ ID NO: 6 - DNA, 22 bp, Artificial sequence
  synthetic
tagcttatcagactgatgttga

SEQ ID NO: 7 - DNA, 22 bp, Artificial sequence
  synthetic
tggctcagttcagcaggaacag

SEQ ID NO: 8 - DNA, 23 bp, Artificial sequence
  synthetic
aaaagtgcttacagtgcaggtag

SEQ ID NO: 9 - DNA, 22 bp, Artificial sequence
  synthetic
tccctgagaccctaacttgtga

SEQ ID NO: 10 - DNA, 21 bp, Artificial sequence
  synthetic
tcacagtgaaccggtctcttt

SEQ ID NO: 11 - DNA, 23 bp, Artificial sequence
  synthetic
ttaatgctaatcgtgataggggt

SEQ ID NO: 12 - DNA, 24 bp, Artificial sequence
  synthetic
tttggcaatggtagaactcacact

SEQ ID NO: 13 - DNA, 22 bp, Artificial sequence
  synthetic
tatggcactggtagaattcact

SEQ ID NO: 14 - DNA, 22 bp, Artificial sequence
  synthetic
ttcaccaccttctccacccagc

SEQ ID NO: 15 - DNA, 23 bp, Artificial sequence
  synthetic
cccagtgtttagactatctgttc

SEQ ID NO: 16 - DNA, 22 bp, Artificial sequence
  synthetic
gtgaaatgtttaggaccactag

SEQ ID NO: 17 - DNA, 22 bp, Artificial sequence
  synthetic
tccttcattccaccggagtctg

SEQ ID NO: 18 - DNA, 22 bp, Artificial sequence
  synthetic
ctgtgcgtgtgacagcggctga

SEQ ID NO: 19 - DNA, 23 bp, Artificial sequence
  synthetic
agctacattgtctgctgggtttc

SEQ ID NO: 20 - RNA, 23 nt, Homo sapiens
uuaaugcuaaucgugauaggggu

SEQ ID NO: 21 - RNA, 24 nt, Homo sapiens
uuuggcaaugguagaacucacacu

SEQ ID NO: 22 - RNA, 22 nt, Homo sapiens
uucaccaccuucuccacccagc

(19) United States
(12) Patent Application Publication
(10) Pub. No.: US 2014/0038194 A1
(43) Pub. Date: Feb. 6, 2014

(54) Method For Early Detection of Lung Cancer

(71) Applicant: The Curators of the University of Missouri, Columbia, MO (US)

(72) Inventors: Michael X. Wang, Columbia, MO (US); Dali Zheng, St. Louis, MO (US)

(21) Appl. No.: 13/958,052

(22) Filed: Aug. 2, 2013

Related U.S. Application Data
(60) Provisional application No. 61/742,161, filed on Aug. 3, 2012.

(51) Int. Cl. C12Q 1/68

(52) U.S. Cl. CPC C12Q 1/6886
USPC 435/6.12

(57) ABSTRACT

The invention provides a blood-based noninvasive early lung cancer detection method, which investigates a panel of miRNA levels in a blood or plasma sample. The panel of miRNA includes miR-17, miR-21, miR-24, miR-106a, miR-125b, miR-128, miR-155, miR-182, miR-183, miR-197, miR-199b, miR-203, miR-205, miR-210, miR-221, and a combination thereof. Preferably, the panel of miRNA may include miR-21, miR-128, miR-155, miR-182, miR-183, and miR-197. The inventive method can not only detect stage I lung cancer patients with high accuracy, but also differentiate between all stages of lung cancer patients and lung cancer-free individuals, metastatic and non-metastatic lung cancer patients and monitor the significant changes of miRNA levels during chemotherapy.

### CROSS-REFERENCE TO RELATED APPLICATIONS

This non-provisional U.S. patent application claims benefit under 35 USC §119(e) of U.S. provisional application No. 61/742,161, filed Aug. 3, 2012.

### INCORPORATION OF SEQUENCE LISTING

The sequence listing that is contained in the file named “52553_{—}124935_SEQ_LIST_ST25.txt”, which is 4,285 bytes in size (measured in operating system MS-Windows), created on Jul. 29, 2013, is filed herewith by electronic submission and incorporated herein by reference in its entirety.

### GRANT STATEMENT

None.

### FIELD OF INVENTION

The present invention relates to the fields of cancer detection and treatment, more specifically, to the early lung cancer detection method employing a panel of microRNAs biomarkers.

### BACKGROUND OF INVENTION

Lung cancer is the leading cause of cancer-related deaths in both men and women worldwide, as well as in the United States [1]. Since there is no validated population-based screening procedure available, most patients with lung cancer are diagnosed at advanced stages with an overall five-year survival rate of only 15% [1]. To improve the outcome of the lung cancer patients, multiple large scale clinical trials to validate screening procedures including chest X-rays, sputum cytology, chest CT, or a combination have been conducted, but none have shown to significantly improve overall mortality over the past 25 years [2]. Recently a large well-designed National Lung Screening Trial (NLST) showed that the low-dose helical CT screening in an older, high-risk population reduced lung cancer mortality by 20% [3, 4]. However, recent studies also indicated that up to 96% of abnormalities by screening CT scans were not lung cancer [4, 5]. The high rate of a false positive result may cause anxiety for the individuals or even lead to unnecessary biopsies or surgery.

Therefore, there is a need to develop a reliable, noninvasive, and cost-effective confirmatory test for detection of lung cancer, which can avoid the over-diagnosis and facilitate the implementation of screening CT scan procedure.

### SUMMARY OF INVENTION

The present invention provides a noninvasive method for early detection of lung cancer, which employs a panel of microRNA (miRNA) biomarkers and detects the miRNA levels in the biological sample, such as plasma. The inventive panel of biomarkers may comprise miR-17, miR-21, miR-24, miR-106a, miR-125b, miR-128, miR-155, miR-182, miR-183, miR-197, miR-199b, miR-203, miR-205, miR-210, miR-221 of plasma. Preferably, the panel of biomarkers may comprise of a combination of multiple biomarkers, such as miR-21, miR-128, miR-155, miR-182, miR-183, and miR-197. With the panel of multiple miRNA biomarkers, the inventive method can not only detect stage I lung cancer patients with enhanced sensitivity and specificity, but also differentiate between metastatic and non-metastatic lung cancer patients and monitor the significant changes of miRNA levels during chemotherapy.

According to one embodiment of the invention, the inventive detection method may be a blood-based test, which comprises the steps of, 1) separating plasma fraction from blood cells shortly after collection of the blood sample from a subject, 2) extracting total RNA from said plasma sample, and 3) detecting miRNA levels in said total RNA by real-time RT-PCR with miRNA-specific primers and their complementary poly(T) reverse primers, whereas said miRNA levels can be compared against the cancer-free control. The miRNA-specific primers may be selected from a group of primers for miR-17, miR-21, miR-24, miR-106a, miR-125b, miR-128, miR-155, miR-182, miR-183, miR-197, miR-199b, miR-203, miR-205, miR-210, miR-221 of plasma. Preferably, miRNA-specific primers comprise miR-21 primer, miR-128 primer, miR-155 primer, miR-182 primer, miR-183 primer, miR-197 primer, or a combination thereof.

In certain embodiments, methods for detecting lung cancer via a blood sample comprising the steps of detecting specific miRNA levels in the blood sample are provided. In certain embodiments of these methods, the miRNAs are selected from a group of cancer associated miRNA biomarkers consisting of miR-17, miR-21, miR-24, miR-106a, miR-125b, miR-128, miR-155, miR-182, miR-183, miR-197, miR-199b, miR-203, miR-205, miR-210, miR-221, and a combination thereof. In certain embodiments, the miRNAs are miR-21, miR-128, miR-155, miR-182, miR-183, miR-197, and a combination thereof. In certain embodiments of the methods, the methods further comprise the steps of: separating plasma fraction from blood cells of the blood sample shortly after collection, extracting total RNA from the separated plasma fraction, and detecting miRNA levels of the extracted total RNA containing miRNAs by real-time RT-PCR with miRNA-specific primers. In certain embodiments of the methods, the detecting step further comprises a SYBR Green-based quantitative RT-PCR assay.

Methods for detecting lung cancer via a blood or plasma sample comprising the steps of: (i) detecting specific miRNA levels in the blood or plasma sample, wherein the miRNAs are selected from a group of cancer associated miRNA biomarkers consisting of miR-21, miR-128, miR-155, miR-182, miR-183, miR-210, miR-221, and miR-197, and a combination thereof; and, (ii) identifying a blood or plasma sample having elevation or devaluation of one or more of the miRNA levels in comparison to a cancer-free control blood or plasma sample, thereby signaling the presence of lung cancer in a patient from which the blood or plasma sample was obtained are also provided. In certain embodiments of the methods, the combination of miR-21, miR-128, miR-155, miR-182, miR-183, miR-210, miR-221, and miR-197 are detected. In certain embodiments of the methods, the combination of miR-21, miR-128, miR-155, miR-182, miR-183, and miR-197 are detected. In certain embodiments of the methods, miR-155, miR-182, and miR-197 are detected, and a blood or plasma sample wherein an increase in the plasma levels of miR-155 and miR-197, but not miR-182 is identified, thereby signaling the presence of metastatic lung cancer in a patient from which the blood or plasma sample was obtained. In certain embodiments, the methods can further comprise the steps of: (i) separating plasma fraction from blood cells of the blood sample shortly after collection; (ii) extracting total RNA from the separated plasma fraction, and (iii) detecting miRNA levels of the extracted total RNA containing miRNAs by real-time RT-PCR with miRNA-specific primers. In certain embodiments of the methods, the detecting step comprises a SYBR Green-based quantitative RT-PCR assay. In certain embodiments the aforementioned methods, elevation of one or more of the miRNA levels in comparison to a cancer-free control sample signals the presence of lung cancer in a patient from which the blood sample was obtained. In certain embodiments, the methods further comprise the steps of: (i) separating a plasma fraction from blood cells of the blood sample shortly after collection; (ii) extracting total RNA from the separated plasma fraction; and, (iii) detecting the specific miRNA levels in the extracted total RNA. In certain embodiments of any of the aforementioned methods, the miRNA levels are detected by a method selected from the group consisting of qRT-PCR, nanopore-based detection, or bead-based detection. Also provided herein are kits for practicing any of the aforementioned methods. Such kits can comprise: (i) reagents for detecting miRNAs selected from a group of cancer associated miRNA biomarkers consisting of miR-21, miR-128, miR-155, miR-182, miR-183, miR-210, miR-221, and miR-197, and a combination thereof and optionally, instructions for their use in signaling the presence of lung cancer or metastatic lung cancer in a patient from which a blood or plasma sample is obtained. The aforementioned methods and kits can be used as the first-line lung cancer screening test in high risk population or a confirmatory test for the low-dose helical CT lung cancer screening procedure.

### DESCRIPTION OF DRAWINGS

FIG. 1: The stability of miRNAs in plasma. Aliquots of two clinical patient plasma samples were subjected to up to six cycles of freeze-thaw (A) or incubated at 4° C. (B) or 37° C. (C) for up to 48 h, or digested with addition of different concentrations of RNase A (D). Then miRNAs were extracted and miR-155 was quantified with qRT-PCR. Data was shown in Ct value. E-F, Pools of extracted total RNA from samples of lung cancer patients (E) and normal controls (F) were subjected to incubation in 37° C. for 2 h with or without RNase A digestion. The levels of miR-155, miR-21 and miR-197 were determined and the data was shown as relative level of miRNAs normalized with the original level.

FIG. 2: MicroRNA selection and screening by qRT-PCR analysis. (A) Heat map clustering of miRNA microarray data from 8 published references (corresponding reference number in brackets), showing certain degree of consistency of the most up-regulated miRNAs (in red) and fewer down-regulated miRNAs (in green) in primary tumor tissues. (B) A total of 15 miRNAs, which were most frequently up-regulated in lung cancer tissues selected from published data (A), were used for screening in 6 plasma samples of lung cancer patients and 6 of normal individual controls by qRT-PCR. Six out of 15 miRNAs (miR-21, 128, 155, 182, 183, 197) were found significantly elevated in plasma from patients with lung cancer compared with controls. Difference of Ct values between lung cancer and control was shown. Note: **, P<0.01; *, P<0.05.

FIG. 3: Sensitivity and specificity of miRNA quantification by a SYBR Green-based real-time RT-PCR (qRT-PCR) method. (A) Standard curves of miR-155, miR-197, and miR-182. The assay showed high sensitivity and a broad dynamic range. (B) Discrimination power (specificity) of the assay on let-7 miRNA family members. Relative detection (%) was calculated based on Ct value difference between perfectly matched and mismatched targets let-7a and let-7b.

FIG. 4: Validation of miR-155, miR-197 and miR-182 in plasma samples of 74 lung cancer patients and 68 cancer-free patient controls. A-C, Scatter plots of plasma levels of miR-155 (A), miR-197 (B) and miR-182 (C) on left panels and receiver operating characteristics (ROC) curve analysis of plasma miR-155 (A), miR-197 (B) and miR-182 (C) on right panels for discriminating lung cancer from control. Ct values were converted to absolute number of miRNA molecules in fmol/L by using a dilution series with known input quantities of synthetic miRNA standard run simultaneously (on the same plate). Concentration of miRNAs was shown as log 10 scale on the Y-axis. (D) Scatter plots of predicted value after logistic regression (left panels) and combination ROC curve (right panels) for discriminating lung cancer from controls. The lines represent the mean value and the bars show the SE. The area under the curve (AUC) and 95% Confidence Interval (CI) are shown in the corresponding charts.

FIG. 5: Correlation of plasma levels of miRNAs and patient clinical status. (A) Box plots of plasma levels of miR-155, miR-197 and miR-182 in lung cancer patients without metastasis (n=44) or with metastasis (n=30). Note: *, P<0.05. (B) Box plots showing plasma levels of miR-155 (left panel), miR-197 (middle panel) and miR-182 (right panel) in control subjects (n=68) and lung cancer patients with different TNM stages (I, n=21; II, n=12; III, n=11; IV, n=30). The lines inside the boxes denote the medians. The boxes mark the interval between 25th and 75th percentiles. The whiskers denote the interval between the 5th and 95th percentiles. Filled circles indicate data points outside the 5th and 95th percentiles.

FIG. 6: The changes of plasma levels of miR-155 (A), miR-197 (B) and miR-182 (C) in patients with lung cancer (n=14) at early and late phases of chemotherapy. Concentration of miRNAs was shown as log 10 scale on the Y-axis. Note: **, P<0.01; *, P<0.05.

FIG. 7: A comparison of the standard curves of miR-221, miR-210 and miR-21 with a real-time RT-PCR miRNA assay (diamonds) and Exigon™ miRNA assay system (squares) (A-C) showed very similar results by these two methods at the standard assay conditions. Both methods demonstrated high sensitivity and a broad dynamic range. The melting curve analysis of miR-155 showed a single peak at 73° C. confirming the specificity of amplified miRNA species with the qRT-PCR miRNA assay method (D).

FIG. 8: Expression of miR21, miR210, miR221 in plasma of normal subjects (the first 8 samples of bar graph) and lung cancer patients (the last 10 samples of bar graph) (A-C) was determined with Exiqon™ miRNA assay system. The levels of these three miRNA in patient samples were significantly higher than those of normal control subjects. In addition, the expression of miR155 was measured in a lung cancer patient at multiple time points during 18 months with invented method (D) showing the levels of miR-155 was correlated with the clinical course of this patient.

### DETAILED DESCRIPTION OF INVENTION

The present invention provides a noninvasive detection method for early detection and diagnosis of lung cancer. The inventive method comprises the step of detecting certain microRNA (miRNA) levels in a biological sample, such as blood sample, whereas the elevation or devaluation of certain miRNA levels in a patient sample in comparison to the cancer-free control may signal the presence of lung cancer.

MicroRNAs (miRNAs) are a class of small non-coding cellular RNAs that regulate gene expression at the posttranscriptional level [6]. A total of 2578 mature miRNAs have been identified in human (on the world wide web at the address “mirbase.org/” ; Release 20: June 2013). Many genes involving in basic biological functions such as cellular proliferation, differentiation, and apoptosis are targets of miRNAs. Accordingly, numerous studies have identified aberrant miRNA expression profiles in many types of human diseases including cancer [7]. Human cancers commonly exhibit an altered expression profile of miRNAs with oncogenic or tumor-suppressive activity [6-8]. Recent studies have revealed that cancer-associated miRNAs play important roles in tumorigenesis; moreover, the miRNAs are highly stable, which allows an efficient isolation from clinical specimens including sputum [9], plasma [10-12], serum [13], and even formalin-fixed paraffin-embedded tissue samples stored for 10 years [14]. Thus, the invention suggests that certain miRNAs may serve as diagnostic and prognostic biomarkers in cancer including lung cancer. These miRNA may be detectible in patient blood samples by using RT-PCR methods (15, 16).

The invention has evaluated the stability of plasma miRNAs in the clinical laboratory setting and the plasma levels of various miRNAs in patients with or without lung cancer and found that certain lung-cancer associated miRNAs of plasma, such as miR-17, miR-21, miR-24, miR-106a, miR-125b, miR-128, miR-155, miR-182, miR-183, miR-197, miR-199b, miR-203, miR-205, miR-210, and miR-221, are acceptable biomarkers, individually or in combination, for early detection and diagnosis of lung cancer. Among the aforementioned miRNAs, six of them, miR-21, miR-128, miR-155, miR-182, miR-183, and miR-197, are found to have significant elevated levels in the lung cancer patient samples comparing to the cancer-free controls. Thus, the panel of six lung-cancer associated miRNAs, individually or in combination, can serve as preferred biomarkers for early detection and diagnosis of lung cancer. Eight of these aforementioned miRNAs, miR-21, miR-128, miR-155, miR-182, miR-183, miR-210, miR-221, and miR-197, are also found to have significant elevated levels in the lung cancer patient blood or plasma samples compared to the cancer-free controls. Thus, in certain embodiments, these eight lung-cancer associated miRNAs (miR-21, miR-128, miR-155, miR-182, miR-183, miR-210, miR-221, and miR-197), used individually, as a collective panel, or in any sub-combination, can also serve as preferred biomarkers for early detection and diagnosis of lung cancer.

The invention also provides an exemplary process employing RT-PCR to determinate lung-cancer-associated miRNA levels in a blood sample. Specifically, the detection process includes the following steps: 1) separating plasma fraction from blood cells shortly after collection of the blood sample from a subject, 2) extracting total RNA from said plasma fraction, and 3) detecting miRNA levels in said total RNA by real-time RT-PCR with miRNA-specific primers and their complementary poly(T) reverse primers.

In the aforementioned separating plasma fraction step, it is recommended to perform the plasma separation shortly (such as, within 1 hour) after blood sample collection to prevent the blood cells' releasing of miRNAs into plasma. Various collection protocols may be employed. For example, the whole blood sample may be collected from a subject (an individual who is at risk of, suspect of, or a carrier of lung cancer) in an EDTA anticoagulant tube. To separate plasma from blood cell fractions, standard separation methods, such as centrifugation technology, may be employed. In an exemplary protocol, centrifugation at 1,600 g for 10 minutes may be adopted. After the separation, the plasma sample may be stored at room temperature, in a refrigerator (4° C.) or freeze condition (between −20° C. to −80° C.) for an extended period of time.

In the aforementioned extracting total RNA step, various standard extraction protocols may be followed. For example, total RNA may be extracted by using miRVana™ PARIS™ kit (Ambion, Austin, Tex.) or miRCURY RNA™ isolation kit (Exiqon A/S, Vedbaek, Denmark) according to the manufacturer's protocol.

In the aforementioned detecting miRNA levels step, though various detecting methods may be used, the present invention adopted the SYBR Green™-based quantitative RT-PCR method (15). In brief, total RNA sample containing miRNA is polyadenylated by poly(A) polymerase (PAP, Ambion, Austin, Tex.) and reverse transcribed to cDNA using SuperScript™ III Reverse Transcriptase (Invitrogen, Carlsbad, Calif.) according to the manufacturer's instructions with a poly(T) adapter universal primer (5′GCGAGCACAGAATTAATACGACTCACTATAGGTTTTTTTTTTTTTTTVN-3′; SEQ ID NO:1) [15]. Real-time PCR can be performed using iQ SYBR Green Supermix™ (Bio-Rad, Hercules, Calif.) with the miRNA specific forward primers (sequences as shown in Table 1) and the sequence complementary to the poly(T) adapter as the reverse primer (5′-GCGAGCACAGAATTAATACGAC-3′ SEQ ID NO:2) in iQ5 Real-time PCR™ system (Bio-Rad). The PCR may be carried out as follows: initial denaturation at 95° C. for 3 min, followed by 50 cycles of 95° C. for 15 s and 60° C. for 40 s and then a dissociation melting curve analysis was conducted to confirm the specificity. Other miRNA detection methods that can be used include, but are not limited to massive parallel signature sequencing (MPSS) (Mineno et al. Nucleic Acids Res 2006, 34:1765-1771), SAGE-based techniques (Cummins et al. Proc Natl Acad Sci USA 2006, 103:3687-3692), microarray-based detection (Yin et al., Trends Biotechnol 2008, 26:70-76), nanopore-based detection or bead-based detection. Nanopore based detection of miRNAs has been described in WO2012/009578 and the corresponding U.S. National Stage Application No. U.S. Ser. No. 13/810,105, the content of which are hereby incorporated by reference in their entireties. Bead-based detection of miRNAs has been described in Biscontin et al. BMC Molecular Biology 2010, 11:44.

### EXAMPLES

The disclosed embodiments are merely representative of the invention, which may be embodied in various forms. Thus, specific structural and functional details disclosed herein are not to be interpreted as limiting.

### Example 1

### Evaluation of miRNAs in Lung Cancer Patient Plasma

To detect miRNA levels, the SYBR Green™-based quantitative RT-PCR method was used (15). In brief, total RNA samples containing miRNA is polyadenylated by poly(A) polymerase (PAP, Ambion, Austin, Tex.) and was reverse transcribed to cDNA using SuperScript™ III Reverse Transcriptase (Invitrogen, Carlsbad, Calif.) according to the manufacturer's instructions with a poly(T) adapter primer (5′GCGAGCACAGAATTAATACGACTCACTATAGGTTTTTTTTTTTTTTTVN-3′; SEQ ID NO:1) [15]. Real-time PCR is performed using iQ SYBR Green Supermix™ (Bio-Rad, Hercules, Calif.) with the miRNA specific forward primers (sequences as shown in Table 1) and the sequence complementary to the poly(T) adapter as the reverse primer (5′-GCGAGCACAGAATTAATACGAC-3′ SEQ ID NO:2) in iQ5 Real-time PCR™ system (Bio-Rad). The PCR was be carried out as follows: initial denaturation at 95° C. for 3 min, followed by 50 cycles of 95° C. for 15 s and 60° C. for 40 s and then a dissociation melting curve analysis was conducted to confirm the specificity.

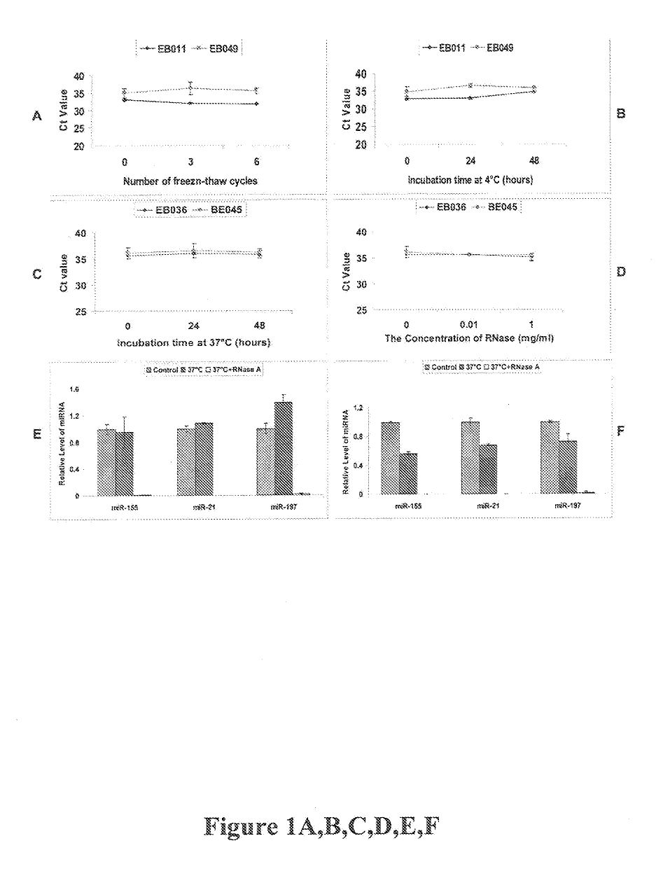

FIG. 1A to FIG. 1F illustrate the stability of plasma miRNAs and purified plasma miRNAs. As shown in FIGS. 1A to 1F, the invention investigated the stability of endogenous miRNAs in plasma samples. Particularly, aliquots of clinical plasma specimens were subjected to 3 or 6 cycles of freeze-thawing or incubated at 4° C. or 37° C. for 24 h and 48 h, and there is minimal or no effect on the levels of miR-155 (FIG. 1A-C) at these conditions.

The stability of plasma miR-155 as well as other miRNAs has been further studied by RNase A digestion. As shown in FIG. 1D, plasma miRNAs show considerable resistance to the enzymatic cleavage of RNase A. However, when purified total RNA was incubated with RNase A, the endogenous miRNAs including miR155, miR21, and miR197 were completely digested, regardless the sample origin, such as the samples from the lung cancer patients (FIG. 1E) or controls (FIG. 1F).

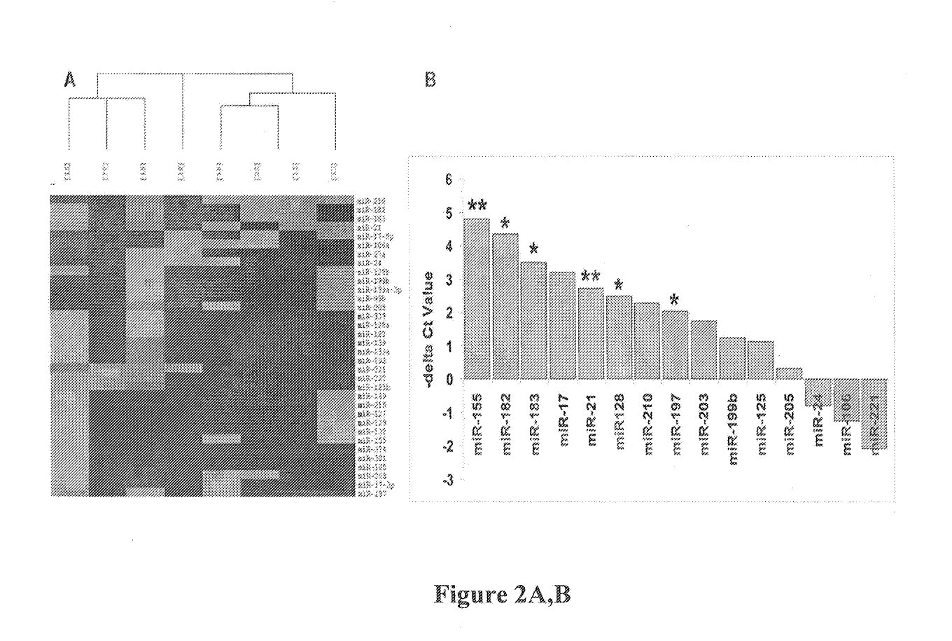

FIG. 2A is a heat map clustering of miRNA microarray data from 8 published references. The inventors have searched for the miRNA microarray data of primary lung cancer based on eight studies of expression profiles of miRNAs in both NSCLC and SCLC tissue types [17-24]. As shown in FIG. 2A, a heat map of the expression profiles of miRNAs has been constructed, and among which 15 miRNAs (miR-17, 21, 24, 106a, 125b, 128, 155, 182, 183, 197, 199b, 203, 205, 210 and 221) have been reported to be most frequently up-regulated in primary lung cancer tissues. However, the levels of these miRNAs in patient blood have not been described.

FIG. 2B summarizes the qRT-PCR analysis of all 15 aforementioned miRNAs levels in plasma samples of lung cancer patients in comparison of the cancer-free controls. Particularly, the inventors determined the levels of all 15 miRNAs in 6 plasma samples from patients with lung cancer and 6 plasma samples from healthy cancer-free individuals. As shown in FIG. 2B, six out of 15 miRNAs (miR-155, 197, 182, 21, 128, and 183) are demonstrated to be significantly elevated in plasma from patients with lung cancer compared with that of controls (P<0.05). Thus, the qRT-PCR analysis demonstrates that the aforesaid miRNAs, especially, miR-155, 197, 182, 21, 128, and 183 are capable of serving as circulating biomarkers for lung cancer detection.

The invention further provides validations of the sensitivity and specificity of the inventive detection method. To define the dynamic range and sensitivity of miRNA quantification by real-time PCR, the synthesized miR-155, miR-197, and miR-182 underwent poly (A) addition reaction and a reverse transcription reaction. The cDNA was diluted by ten orders of magnitude and subjected to real-time PCR.

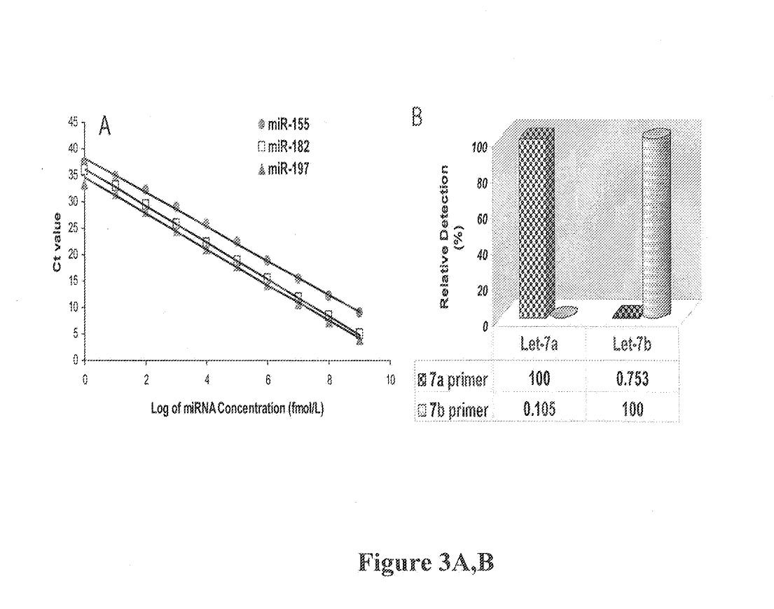

FIG. 3A shows standard curves of miR-155, miR-197, and miR-182. As shown in FIG. 3A, the real-time RT-PCR assay demonstrates excellent linearity between the log of miRNA concentration and cycle threshold (Ct) value, indicating that the assay has a dynamic range of 9-10 logs and is thus capable of detecting as few as 5 copies of miRNA per PCR reaction, and the correlation coefficient was 0.998 for miR-155, 0.998 for miR-197, and 0.999 for miR-182. Thus, the method adopted is proven to have high sensitivity and a broad dynamic range.

TABLE 1
The sequence of synthetic primers and
single-stranded miRNAs

| Primers and | | |
|---|---|---|
| miRNAs | Primer Sequence (SEQ ID NO:) | |
| Let-7a primer | TGAGGTAGTAGGTTGTATAGTT | (SEQ ID NO: 3) |
| Let-7b primer | TGAGGTAGTAGGTTGTGTGGTT | (SEQ ID NO: 4 |
| miR-17 primer | CAAAGTGCTTACAGTGCAGGTAG | (SEQ ID NO: 5) |
| miR-21 primer | TAGCTTATCAGACTGATGTTGA | (SEQ ID NO: 6) |
| miR-24 primer | TGGCTCAGTTCAGCAGGAACAG | (SEQ ID NO: 7) |
| miR-106a primer | AAAAGTGCTTACAGTGCAGGTAG | (SEQ ID NO: 8) |
| miR-125b primer | TCCCTGAGACCCTAACTTGTGA | (SEQ ID NO: 9) |
| miR-128 primer | TCACAGTGAACCGGTCTCTTT | (SEQ ID NO: 10) |
| miR-155 primer | TTAATGCTAATCGTGATAGGGGT | (SEQ ID NO: 11) |
| miR-182 primer | TTTGGCAATGGTAGAACTCACACT | (SEQ ID NO: 12) |
| miR-183 primer | TATGGCACTGGTAGAATTCACT | (SEQ ID NO: 13) |
| miR-197 primer | TTCACCACCTTCTCCACCCAGC | (SEQ ID NO: 14) |
| miR-199b primer | CCCAGTGTTTAGACTATCTGTTC | (SEQ ID NO: 15) |
| miR-203 primer | GTGAAATGTTTAGGACCACTAG | (SEQ ID NO: 16) |
| miR-205 primer | TCCTTCATTCCACCGGAGTCTG | (SEQ ID NO: 17) |
| miR-210 primer | CTGTGCGTGTGACAGCGGCTGA | (SEQ ID NO: 18) |
| miR-221 primer | AGCTACATTGTCTGCTGGGTTTC | (SEQ ID NO: 19) |
| miR-155 RNA | UUAAUGCUAAUCGUGAUAGGGGU | (SEQ ID NO: 20) |
| miR-182 RNA | UUUGGCAAUGGUAGAACUCACACU | (SEQ ID NO: 21) |
| miR-197 RNA | UUCACCACCUUCUCCACCCAGC | (SEQ ID NO: 22) |

Refer to FIG. 3B, which illustrates discrimination power of the assay on let-7 miRNA family members. To determine the specificity of real-time PCR assay for miRNAs, the synthesized two Let-7 miRNA family members, Let-7a and Let-7b, were detected with primers specific for Let-7a and Let-7b, respectively. Relative detection efficiency was calculated from Ct value differences between perfectly matched and mismatched targets, assuming 100% efficiency for the perfect match. Very low levels of non-specific amplification (less than 1%) were observed (FIG. 3B), similar to the method of stem-loop RT-PCR [16]. These results suggest that the SYBR Green-based quantitative RT-PCR assay is highly specific and can discriminate miRNAs that differ by as few as two nucleotides.

Moreover, the invention has provided clinical validation of miRNA biomarkers in patient plasma samples. To evaluate the diagnostic value of the aforementioned lung-cancer associated miRNAs, the levels of miR-155, 197, and 182 have been measured on a total 142 plasma samples, including 74 samples of lung cancer patients at various stages and 68 samples of cancer-free age-matched controls (Table 2).

TABLE 2
Characteristics of lung cancer patients

| | Lung cancer (n = 74) |
|---|---|
| Age | Ave 64.2 (SD 10.9, range from 43-87) |
| Sex | |
| Male | 40 |
| Female | 34 |
| Tumor | |
| SCLC | 17 |
| NSCLC | 48 |
| AC | 18 |
| SC | 23 |
| LC | 7 |
| Other | 9 |
| Stages | |
| Stage I | 21 |
| Stage II | 12 |
| Stage III | 11 |
| Stage IV | 30 |
| Metastasis | |
| YES | 30 |
| NO | 44 |

Abbreviations:
SCLC, small cell lung cancer; NSCLC, non-small cell lung cancer;
AC, adenocarcinoma; SC, squamous cell carcinoma; Ave, average;
Controls, n = 68 including male 37, female 31.
Average age of 61.2 with SD 14.0, ranged from 36 to 93 years old.

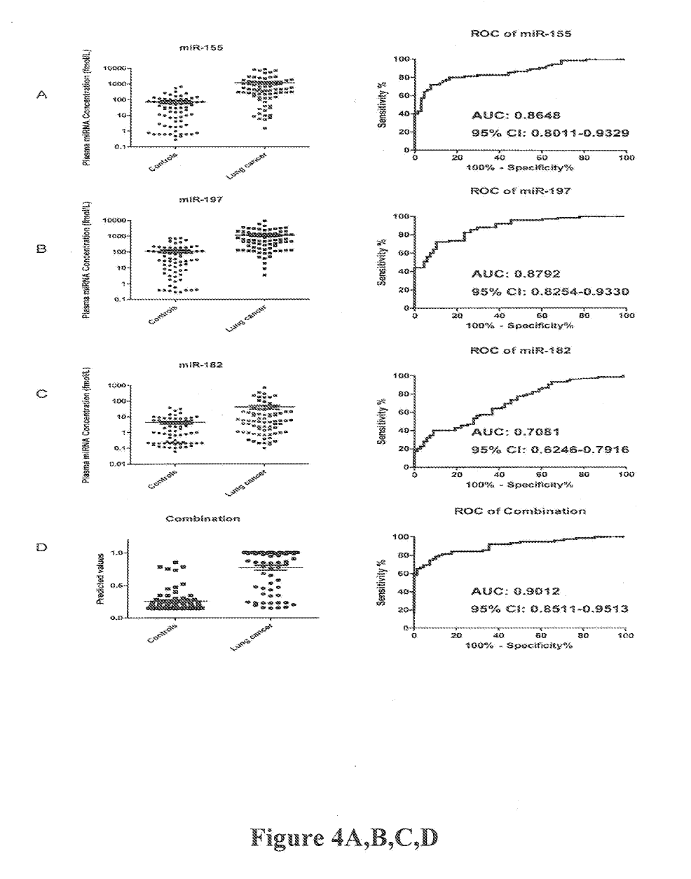

Refer to FIGS. 4A to 4D, which are scatter plots and receiver operation characteristics (ROC) curves of plasma levels of selected miRNAs. Because there is no consensus on the use of housekeeping miRNA for plasma qRT-PCR quantification [10-13, 20, 25], the inventors decided to measure miRNA expression levels converted to absolute concentration in fmol/L by using a dilution series of known input quantities of synthetic miRNA run simultaneously (on the same plate) as the experimental samples. The levels of the three miRNAs (miR-155, miR197, and miR182) are significantly higher, in average, in the lung cancer cohort than in controls (P<0.001), corresponding to an average fold-change of 17.08, 10.46 and 9.74, respectively (FIG. 4A-C, left panels). ROC curve analysis shows that all of these three miRNAs could differentiate lung cancer from controls with an AUC of 0.8648 for miR-155 (95% CI: 0.8011-0.9329, P<0.001), 0.8792 for miR-197 (95% CI: 0.8254-0.9330, P<0.001), and 0.7081 for miR-182 (95% CI: 0.6246-0.7916, P<0.001), respectively (FIG. 4A-C, right panels). The predicted values of logistic regression showed significant difference between these two groups (FIG. 4D, left panel, P<0.001) and combination ROC analysis revealed increased AUC value to 0.9012 (FIG. 4D, right panel, 95% CI: 0.8511-0.9513, P<0.001) with 81.33% sensitivity and 86.76% specificity.

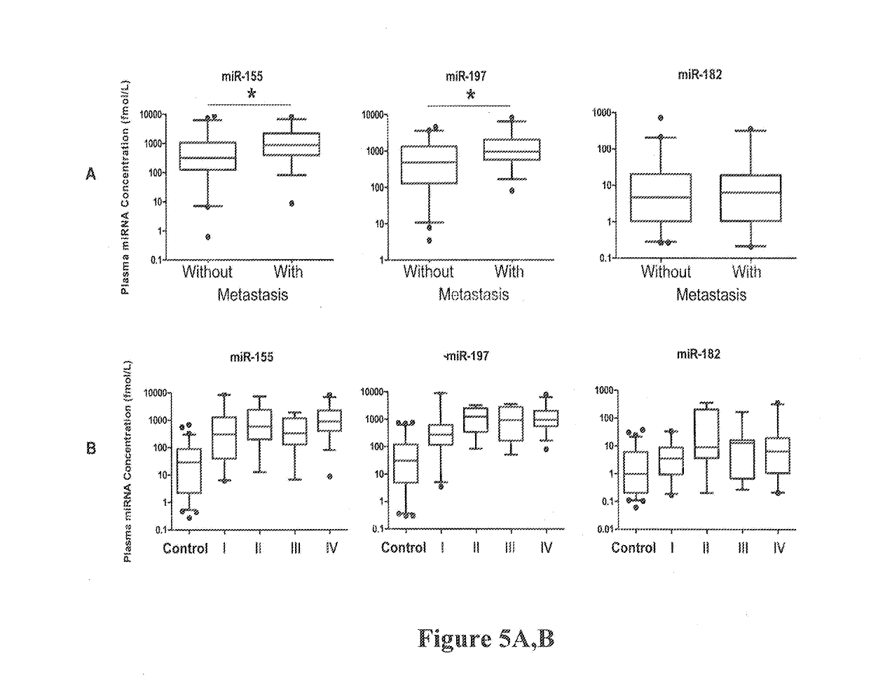

The invention has also evaluated relationships between plasma levels of lung-cancer-associated miRNAs and patient clinical status. Refer to FIGS. 5A and 5B, which examine the correlation between the plasma levels of miR-155, miR-197, and miR-182 with patient clinical parameters. No significant association has been found between the levels of these three miRNAs and sex, age, smoke history, nodal status or histological types of tumor, respectively, while the plasma levels of miR-155 and miR-197, but not miR-182, in the patients with metastasis were significantly higher than in those without metastasis (P<0.05, FIG. 5A).

As shown in FIG. 5B, the levels of all 3 miRNAs are not significantly different between the stages, however, each of the 4 stages including stage I patients has significantly elevated plasma miR-155, miR-197, and miR-182 levels when compared with the cancer-free controls (P<0.01).

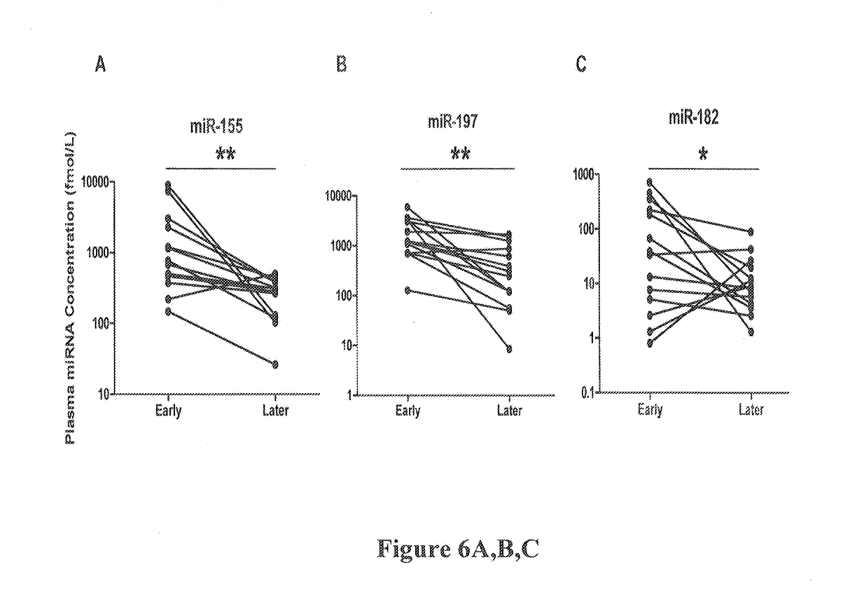

Refer to FIGS. 6A to 6C, which illustrate the changes of plasma levels of 3 miRNAs in patients undergoing lung cancer chemotherapy. Particularly, to explore the value of these 3 miRNAs in monitoring of treatment effectiveness by chemotherapy, their plasma levels have been measured in a set of 14 patients with lung cancer in early and late phases of chemotherapy. The results suggest that the levels of miRNA-155, miR-197, and miR-182 have been significantly reduced after treatment (P<0.01 for miR-155 and miR-197, P<0.05 for miR-182) in the majority of responsive patients. Thus, the inventive method may be developed to monitor significant changes of miRNA levels during chemotherapy to indicate the effectiveness of chemotherapy on an individual patient

### Example 2

### Further Validation of miRNA Assay Methods

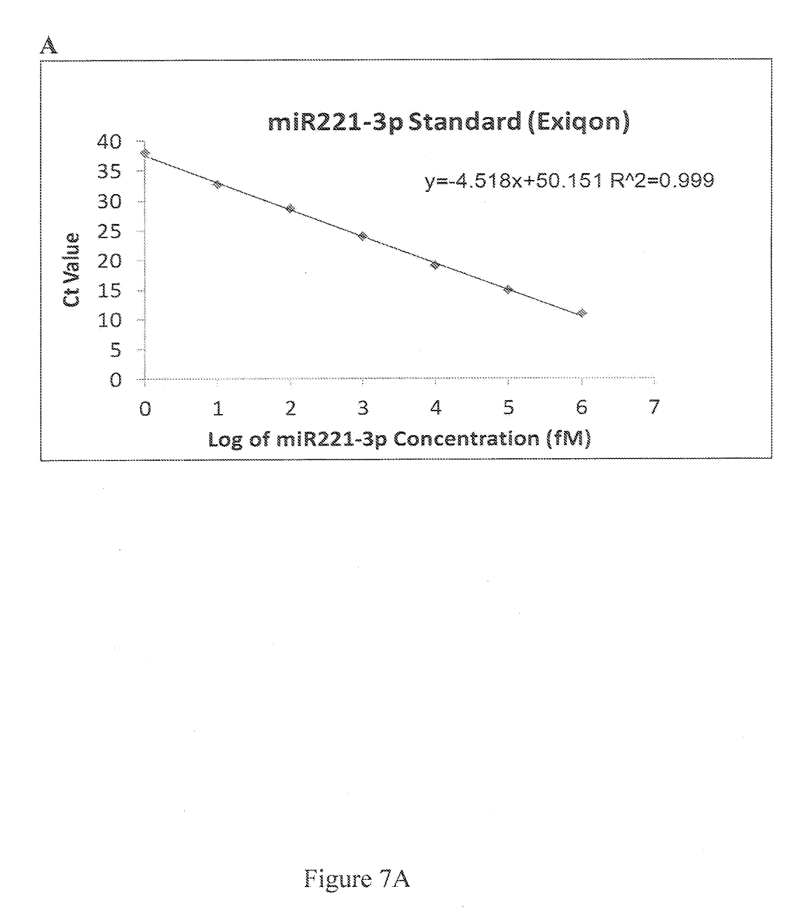

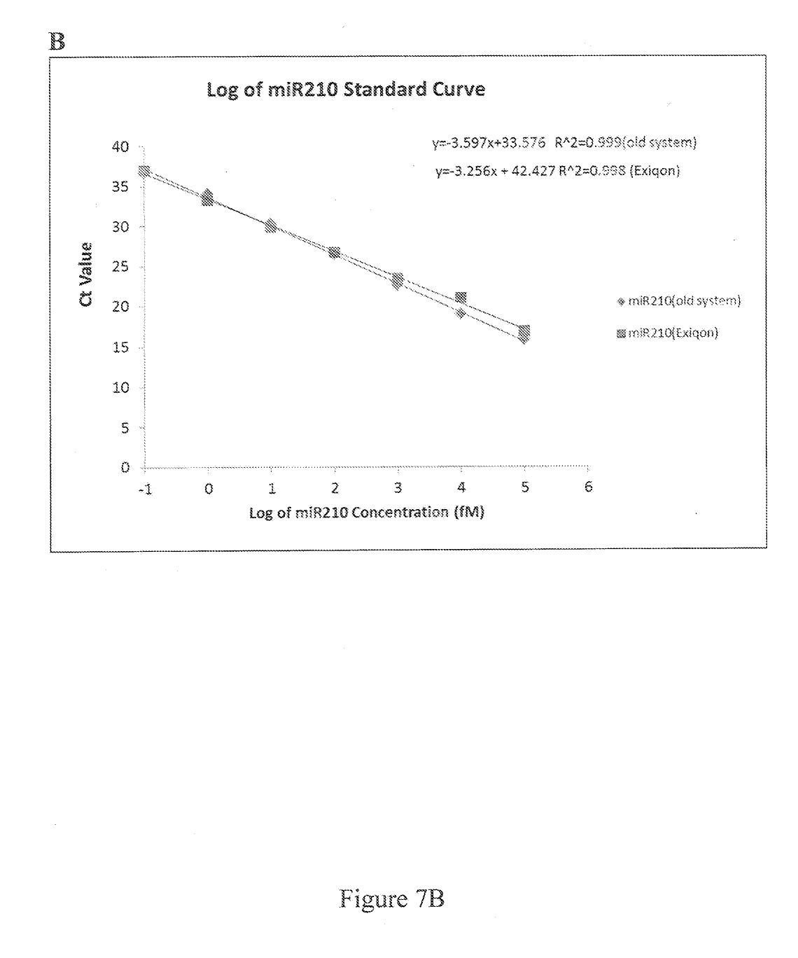

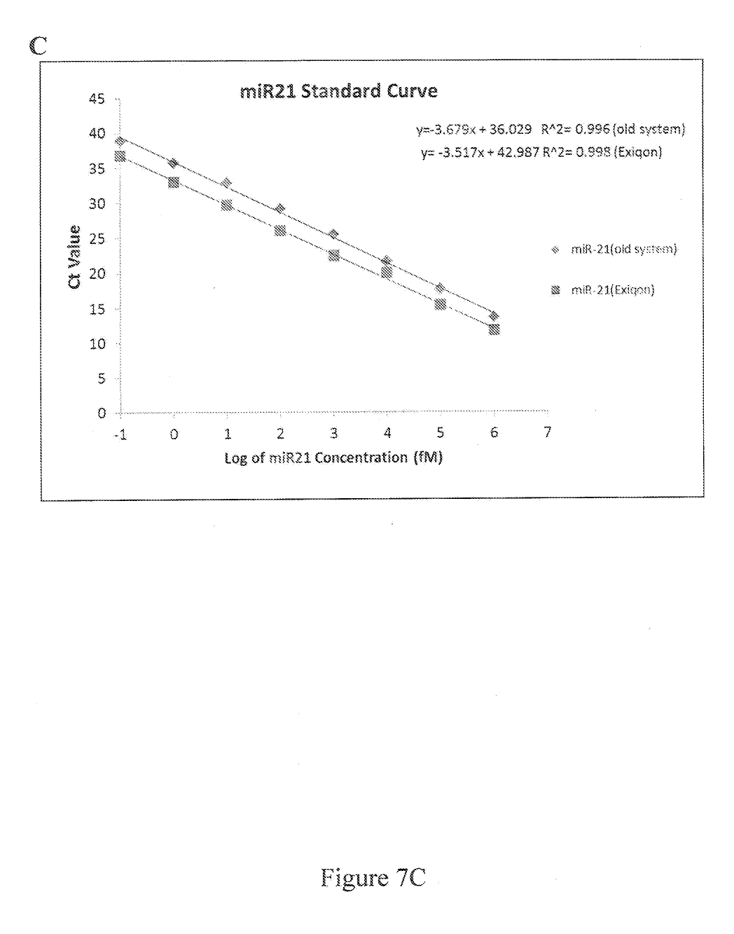

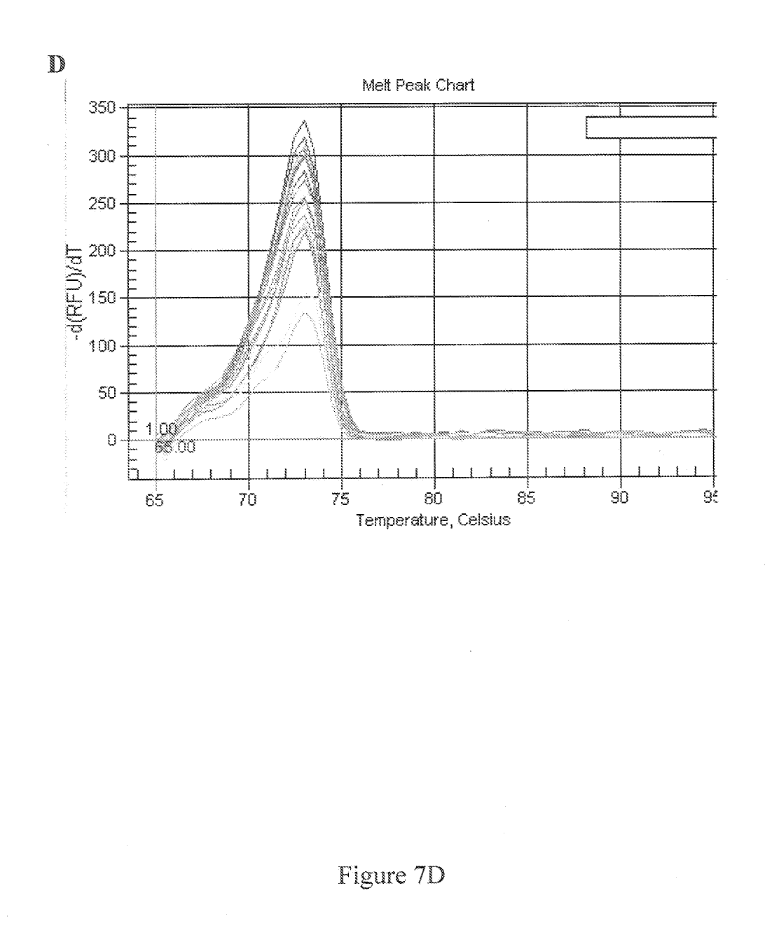

To further validate the miRNA assay method, Exiqon miRCURY LNA™ Universal RT microRNA PCR system including miRCURY RNA™ isolation kit (Prod. No. 30012), Universal cDNA Synthesis Kit II (Prod. No. 203301), miRCURY LNA™ Universal RT microRNA PCR (Product No. 203203), ExiLENT SYBR® Green master mix (Product No. 203420), hsa-miR-221-3p, LNA PCR primer set (Prod. No. 204532), hsa-miR-21-5p, LNA PCR primer set (Prod. No. 204230), hsa-miR-210, and LNA PCR primer set (Prod. No. 204333) was used to measure miRNAs according to manufacturer's instruction (FIGS. 7 A-C). The standard synthesized miR-221, miR-210 and miR-21 (Integrated DNA Technologies, Coralville, Iowa) were used as the templates in respective reactions comparing the PCR assays.

As shown in FIG. 7A, the Exigon™ miRNA assay system demonstrates excellent linearity between the log of miRNA concentration (fM) and cycle threshold (Ct) value, indicating that the assay has a dynamic range of 7 logs and is thus capable of detecting as few as 300 copies of miRNA, and the correlation coefficient was 0.999 for miR-221. Similarly, the standard curves of miR-210 and miR-21 with both the qRT_PCR assay of Example 1 and the Exigon™ methods (FIG. 7 B-C) demonstrated the dynamic linear range of 7-8 logs and the correlation coefficients were 0.996-0.998 although Exiqon miRNA assay system was more sensitive for miR-21 assay (2.5 Ct difference) than invented method. The melting curve analysis (FIG. 7D) showed a specific peak for miR-155 indicating the specific amplification. This step should be included in each assay to confirm the assay specificity especially in SYBR Green based PCR methods.

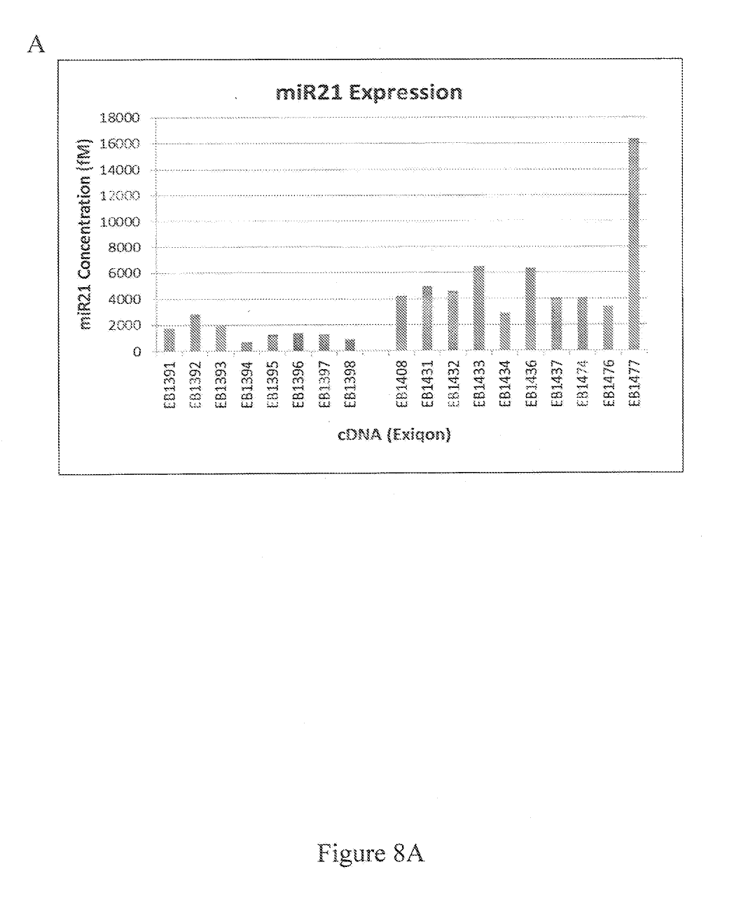

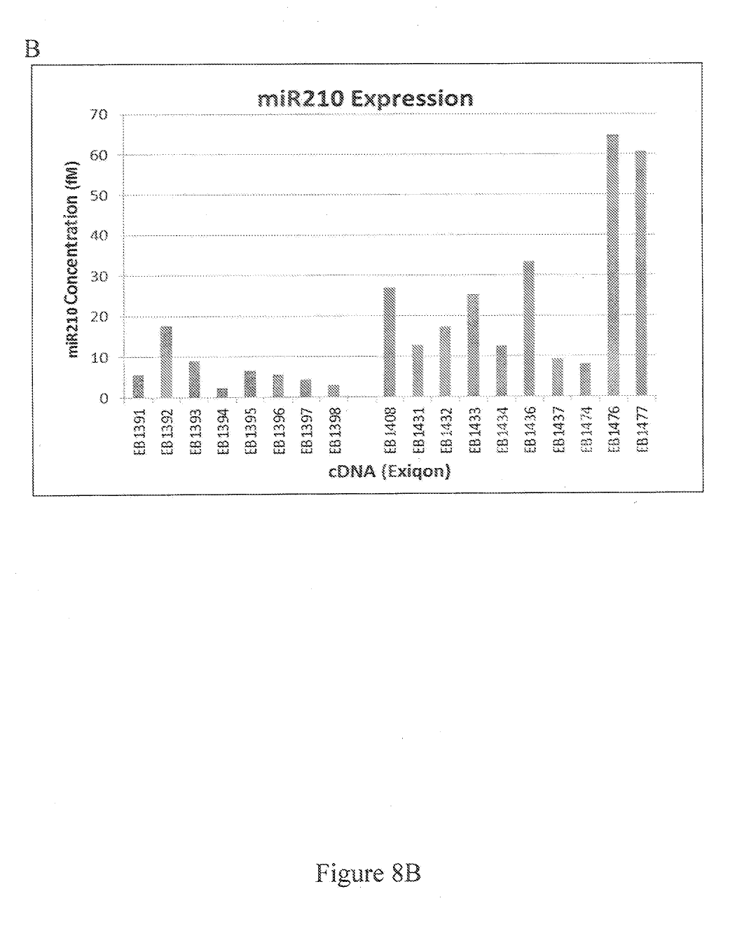

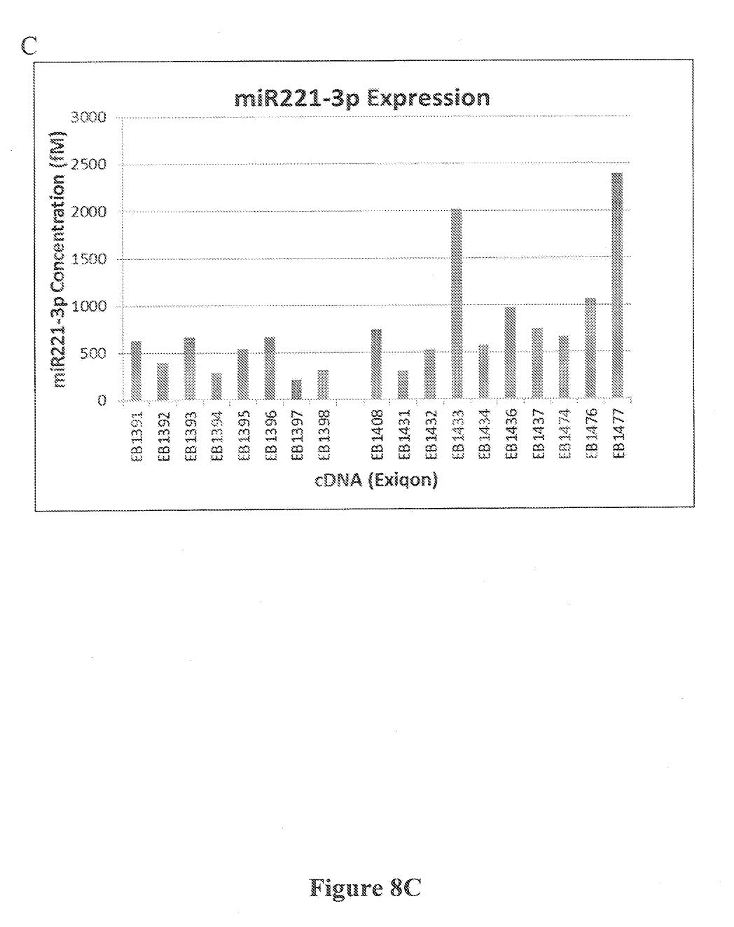

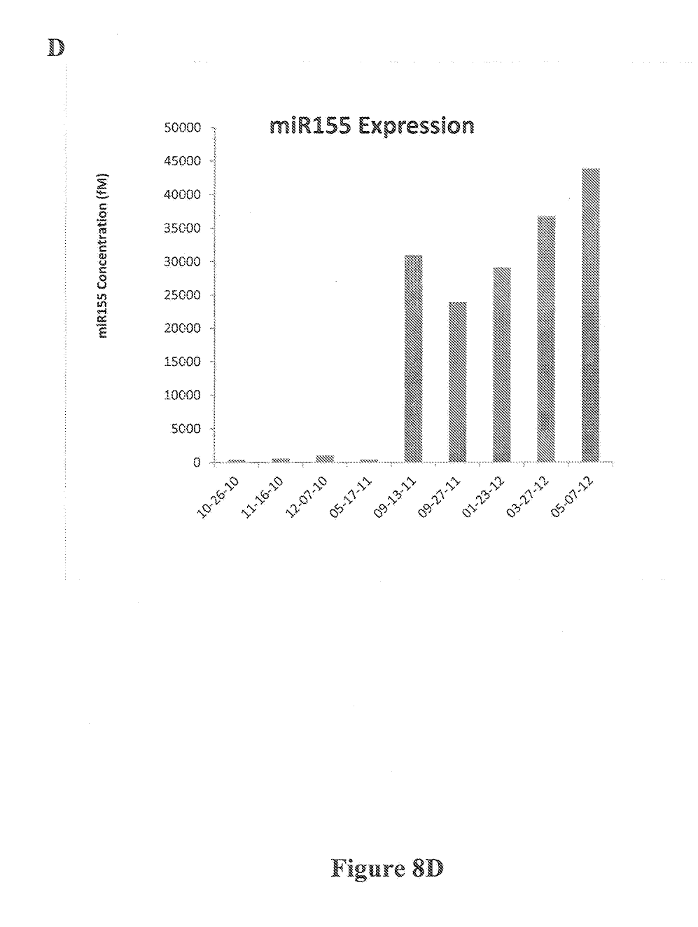

Additional microRNA biomarkers including miR21, miR210 and miR221 were validated in clinical samples (FIG. 8 A-C). The expression of miR21, miR210 and miR221 in plasma samples of the 8 normal subjects (EB1391, EB1392, EB1393, EB1394, EB1395, EB1396, EB1397 and EB1398) and 10 lung cancer patients (EB1408, EB1431, EB1432, EB1433, EB1434, EB1436, EB1437, EB1474, EB1476 and EB1477) was determined with the Exiqon miRNA assay system. The average of all three miRNA levels was significantly higher in lung cancer patients than these of the normal controls. Additionally, the expression of miR-155 in a single lung cancer patient at multiple time points was determined with the invented microRNA assay method (FIG. 8D). The miR-155 was elevated when a passible metastatic lesion at the lumber area was identified with a CT scan on 09-13-11 in this patient. The primary lung cancer and the passible metastatic lesion persisted during the course of chemotherapy indicating miR-155 may be as a biomarker for lung cancer progression and monitory of therapy effectiveness.

While the invention has been described in connection with specific embodiments thereof, it will be understood that the inventive device is capable of further modifications. This patent application is intended to cover any variations, uses, or adaptations of the invention following, in general, the principles of the invention and including such departures from the present disclosure as come within known or customary practice within the art to which the invention pertains and as may be applied to the essential features herein before set forth. All such variations, uses, or adaptations apparent to those skilled in the art are deemed to be within the spirit, scope and concept of the invention as defined by the appended claims.

### REFERENCES

1. Siegel R, Ward E, Brawley O, Jemal A. Cancer statistics 2011, The impact of eliminating socioeconomic and racial disparities on premature cancer deaths. CA Cancer J Clin. 2011; 61:212-236.
2. Bach P B, Jett J R, Pastorino U, Tockman M S, Swensen S J, Begg C B. Computed tomography screening and lung cancer outcomes. JAMA 2007; 297: 953-961.
3. National Lung Screening Trial Research Team. The National Lung Screening Trial overview and study design. Radiology 2011; 258:243-253.
4. Team, Aberle D R, Adams A M, Berg C D, Black W C, Clapp J D, Fagerstrom R M, Gareen I F, Gatsonis C, Marcus P M, Sicks J D: Reduced Lung-Cancer Mortality with Low-Dose Computed Tomographic Screening. N Engl J Med. 2011 Aug. 4; 365(5):395-409.
5. Barba M, Felsani A, Rinaldi M, Giunta S, Malorni W, Paggi M G. Reducing the risk of overdiagnosis in lung cancer: A support from molecular biology. J Cell Physiol. 2011; 226:2213-2214.
6. Garzon R, Calin G A, and Croce C M. MicroRNAs in Cancer. Annu Rev Med 2009; 60:167-179.
7. Davalos V and Esteller M. MicroRNAs and cancer epigenetics: a macroevolution. Curr Opin Oncol 2010; 22:35-45.
8. Lu J, Getz G, Miska E A, Alvarez-Saavedra E, Lamb J et al. MicroRNA expression profiles classify human cancers. Nature 2005; 435:834-838.
9. Xie Y, Todd N W, Liu Z, Zhan M, Fang H. Altered miRNA expression in sputum for diagnosis of non-small cell lung cancer. Lung Cancer 2010; 67:170-176.
10. Mitchell P S, Parkin R K, Kroh E M, Fritz B R, Wyman S K et al. Circulating microRNAs as stable blood-based markers for cancer detection. Proc Natl Acad Sci USA 2008; 105:10513-10518.
11. Tsujiura M, Ichikawa D, Komatsu S, Shiozaki A, Takeshita H, Kosuga T, et al. Circulating microRNAs in plasma of patients with gastric cancers. Br J Cancer 2010; 102:1174-1179.
12. Huang Z, Huang D, Ni S, Peng Z, Sheng W, Du X. Plasma microRNAs are promising novel biomarkers for early detection of colorectal cancer. Int J Cancer 2010; 127:118-126.
13. Chen X, Ba Y, Ma L, Cai X, Yin Y, Wang K, Guo J et al. Characterization of microRNAs in serum: a novel class of biomarkers for diagnosis of cancer and other diseases. Cell Res 2008; 18:997-1006.
14. Xi Y, Nakajima G, Gavin E, Morris C G, Kudo K et al. Systematic analysis of microRNA expression of RNA extracted from fresh frozen and formalin-fixed paraffin-embedded samples. RNA 2007; 13:1668-1674.
15. Shi R, Chiang V L. Facile means for quantifying microRNA expression by real-time PCR. Biotechniques 2005; 39:519-525.
16. Chen C, Ridzon D A, Broomer A J, Zhou Z, Lee D H et al. Real-time quantification of microRNAs by stem-loop RT-PCR. Nucleic Acids Res 2005; 33: e179.
17. Yu S L, Chen H Y, Chang G C, Chen C Y, Chen H W et al. MicroRNA signature predicts survival and relapse in lung cancer. Cancer Cell 2008; 13:48-57.
18. Volinia S, Calin G A, Liu C G, Ambs S, Cimmino A et al. A microRNA expression signature of human solid tumors defines cancer gene targets. Proc Natl Acad Sci USA 2006; 103: 2257-2261.
19. Yanaihara N, Caplen N, Bowman E, Seike M, Kumamoto K et al. Unique microRNA molecular profiles in lung cancer diagnosis and prognosis. Cancer Cell 2006; 9: 189-198.
20. Peltier H J, Latham G J. Normalization of microRNA expression levels in quantitative RT-PCR assays: identification of suitable reference RNA targets in normal and cancerous human solid tissues. RNA 2008; 14: 844-852.
21. Garofalo M, Quintavalle C, Di Leva G, Zanca C, Romano G et al. MicroRNA signatures of TRAIL resistance in human non-small cell lung cancer. Oncogene 2008; 27:3845-3855.
22. Cho W C, Chow A S, Au J S. Restoration of tumour suppressor hsa-miR-145 inhibits cancer cell growth in lung adenocarcinoma patients with epidermal growth factor receptor mutation. Eur J Cancer 2009; 45:2197-2206.
23. Raponi M, Dossey L, Jatkoe T, Wu X, Chen G et al. MicroRNA classifiers for predicting prognosis of squamous cell lung cancer. Cancer Res 2009; 69:5776-5783.
24. Miko E, Czimmerer Z, Csánky E, Boros G, Buslig J et al. Differentially expressed microRNAs in small cell lung cancer. Exp Lung Res 2009; 35:646-664.

What claimed is:

1. A method for detecting lung cancer via a blood or plasma sample comprising the steps of:
(i) detecting specific miRNA levels in said blood or plasma sample, wherein said miRNAs are selected from a group of cancer associated miRNA biomarkers consisting of miR-21, miR-128, miR-155, miR-182, miR-183, and miR-197, and a combination thereof; and,
(ii) identifying a blood or plasma sample having elevation or devaluation of one or more of said miRNA levels in comparison to a cancer-free control blood or plasma sample, thereby signaling the presence of lung cancer in a patient from which the blood or plasma sample was obtained.

2. The method of claim 1, wherein the combination of miR-21, miR-128, miR-155, miR-182, miR-183, and miR-197 are detected.

3. The method of claim 1, wherein miR-155, miR-182, and miR-197 are detected, and a blood or plasma sample wherein an increase in the plasma levels of miR-155 and miR-197, but not miR-182 is identified, thereby signaling the presence of metastatic lung cancer in a patient from which the blood or plasma sample was obtained.

4. The method of claim 1, further comprising the steps of:
i) separating plasma fraction from blood cells of said blood sample shortly after collection,
ii) extracting total RNA from said separated plasma fraction, and
iii) detecting miRNA levels of said extracted total RNA containing miRNAs by real-time RT-PCR with miRNA-specific primers.

5. The method of claim 4, wherein said detecting step further comprising a SYBR Green-based quantitative RT-PCR assay.

6. The method of claim 1, wherein elevation of one or more of said miRNA levels in comparison to a cancer-free control sample signals the presence of lung cancer in a patient from which the blood sample was obtained.

7. The method of claim 1, further comprising the steps of
(i) separating plasma fraction from blood cells of said blood sample shortly after collection,
(ii) extracting total RNA from said separated plasma fraction; and
(iii) detecting said specific miRNA levels in said extracted total RNA.

8. The method of claim 1, further comprising the steps of:
(i) extracting total RNA from said plasma; and,
(ii) detecting said specific miRNA levels in said extracted total RNA.
